(19) United States
(12) Patent Application Publication
(10) Pub. No.: US 2015/0182123 A1
(43) Pub. Date: Jul. 2, 2015

(54) OBJECT INFORMATION ACQUIRING APPARATUS AND CONTROL METHOD FOR THE OBJECT INFORMATION ACQUIRING APPARATUS

(71) Applicant: CANON KABUSHIKI KAISHA, Tokyo (JP)

(72) Inventor: Akira Sato, Kawasaki-shi (JP)

(21) Appl. No.: 14/423,874

(22) PCT Filed: Sep. 17, 2013
(86) PCT No.: PCT/JP2013/005491

§ 371 Date: Feb. 25, 2015

(30) Foreign Application Priority Data
Sep. 21, 2012 (JP) 2012-208123

(51) Int. Cl. A61B 5/00

(52) U.S. Cl. CPC A61B 5/0095; A61B 5/4312

(57) ABSTRACT

An object information acquiring apparatus comprises a first light source configured to generate first irradiation light; a second light source configured to generate second irradiation light; a first illumination optical system configured to guide the first irradiation light to the object; a second illumination optical system configured to guide the second irradiation light to the object; a light sensor unit configured to acquire an intensity signal of the reflected second irradiation light on the object; and a control device configured to determine irradiation propriety of the first irradiation light on the basis of the intensity signal of the reflected light. The second illumination optical system shares at least a part of an optical member with the first illumination optical system.

### TECHNICAL FIELD

The present invention relates to a technique for observing components or shapes on the surface and inside of an object.

### BACKGROUND ART

There has been devised a technique for irradiating laser light on a living organism to generate an ultrasound wave (a photoacoustic wave) due to the laser irradiation on the inside of the living organism and analyzing the photoacoustic wave to analyze the structure or the situation on the surface and the inside of the living organism. This is called photoacoustic measurement. Since a noninvasive test can be performed, there is also a trend for diverting the technique to medical treatment to perform a test of the inside of a human body. There is known X-ray mammography for the purpose of a test and a diagnosis of breast cancer. As disclosed in NPL 1, a photoacoustic measurement apparatus of a manual scanning type for the purpose of a breast cancer examination has also been developed.

Measurement light represented by laser light decays when the measurement light is propagated and diffused on the inside of an object. Therefore, in order to cause the measurement light to reach the depth of a living organism of the object, it is necessary to irradiate a sufficient amount of light on the surface of the object. In general, the measurement light has high energy. Therefore, in the photoacoustic measurement apparatus of the manual scanning type, irradiation of the measurement light on regions other than the object has to be prevented.

For example, PTL 1 discloses a technique for, in a general laser treatment device, detecting contact of a skin and an instrument and performing irradiation of laser light. By applying the technique described in PTL 1 to the photoacoustic measurement apparatus, it is possible to prevent irradiation of the measurement light on regions other than the object.

### CITATION LIST

### Patent Literature

[PTL 1] Japanese Patent Application Laid-Open No. H9-253224

### Non Patent Literature

[NPL 1] S. A. Ermilov et al., Development of laser optoacoustic and ultrasonic imaging system for breast cancer utilizing handheld array probes, Photons Plus Ultrasound: Imaging and Sensing 2009, Proc. of SPIE vol. 7177, 2009

### SUMMARY OF INVENTION

### Technical Problem

As explained above, in order to prevent irradiation of the measurement light on regions other than the object, it is conceivable to use means such as a contact sensor for detecting that a probe is in contact with the object. However, if the contact is detected to perform irradiation control for irradiation light, the probe and the object have to be caused to completely adhere to each other. Therefore, measurement cannot be performed for an object having unevenness on the surface. It is desirable to enable measurement even if the probe and the object do not completely adhere to each other as long as irradiation light leaking from a space between the probe and the object is at a level safe for the human body.

In view of the problems, it is an object of the present invention to provide an object information acquiring apparatus that can perform irradiation control for the irradiation light after determining whether the irradiation light leaking from the space between the probe and the object is at the safe level.

### Solution to Problem

In order to solve the problems, the present invention in its one aspect provides an object information acquiring apparatus that irradiates light on an object, receives an acoustic wave generated in the object, and acquires information concerning an inside of the object on the basis of the acoustic wave, comprising a first light source configured to generate first irradiation light to be irradiated on the object, this light being emitted to generate an acoustic wave from the object; a second light source configured to generate second irradiation light to be irradiated on the object; a first illumination optical system connected to the first light source and configured to guide the first irradiation light to the object; a second illumination optical system connected to the second light source and configured to guide the second irradiation light to the object; a light sensor unit including a light sensor and configured to acquire an intensity signal of reflected light on the object resulting from the reflection of the second irradiation light irradiated on the object; and a control device configured to determine irradiation propriety of the first irradiation light on the basis of the intensity signal of the reflected light acquired by the light sensor unit, wherein the second illumination optical system shares at least a part of an optical member with the first illumination optical system.

The present invention in its another aspect provides a control method for an object information acquiring apparatus that irradiates light on an object, receives an acoustic wave generated in the object, and acquires information concerning an inside of the object on the basis of the acoustic wave, comprising a step of generating first irradiation light to be irradiated on the object, with this light being emitted to generate an acoustic wave from the object, and of irradiating the first irradiation light on the object through an illumination optical system; a step of generating second irradiation light to be irradiated on the object and irradiating the second irradiation light on the object through at least a part of the illumination optical system through which the first irradiation light is irradiated; and a step of acquiring, by a light sensor, an intensity signal of reflected light on the object resulting from the reflection of the second irradiation light irradiated on the object, and of determining propriety of irradiation of the first irradiation light on the basis of the acquired intensity signal.

### Advantageous Effects of Invention

According to the present invention, it is possible to provide an object information acquiring apparatus that can perform irradiation control for the irradiation light after determining whether the irradiation light leaking from the space between the probe and the object is at the safe level.

Further features of the present invention will become apparent from the following description of exemplary embodiments with reference to the attached drawings.

### BRIEF DESCRIPTION OF DRAWINGS

FIG. 1 is a block diagram schematically showing the configuration of a photoacoustic measurement apparatus according to a first embodiment.

FIG. 2 is a diagram for explaining a method of merging optical axes of laser light and evaluation light.

FIG. 3 is a processing flowchart of the photoacoustic measurement apparatus according to the first embodiment.

FIG. 4 is a diagram for explaining a pattern of contact of a probe with an object.

FIG. 5 is a diagram showing a relation between the distance to the object and the intensity of reflected light.

FIG. 6 is a light emission timing chart of laser light and LED light.

FIG. 7 is a block diagram schematically showing the configuration of a photoacoustic measurement apparatus according to a second embodiment.

### DESCRIPTION OF EMBODIMENTS

Embodiments of the present invention are explained in detail below with reference to the drawings. Note that, in principle, the same components are denoted by the same reference numerals and signs and explanation of the components is omitted.

### First Embodiment

### System Configuration

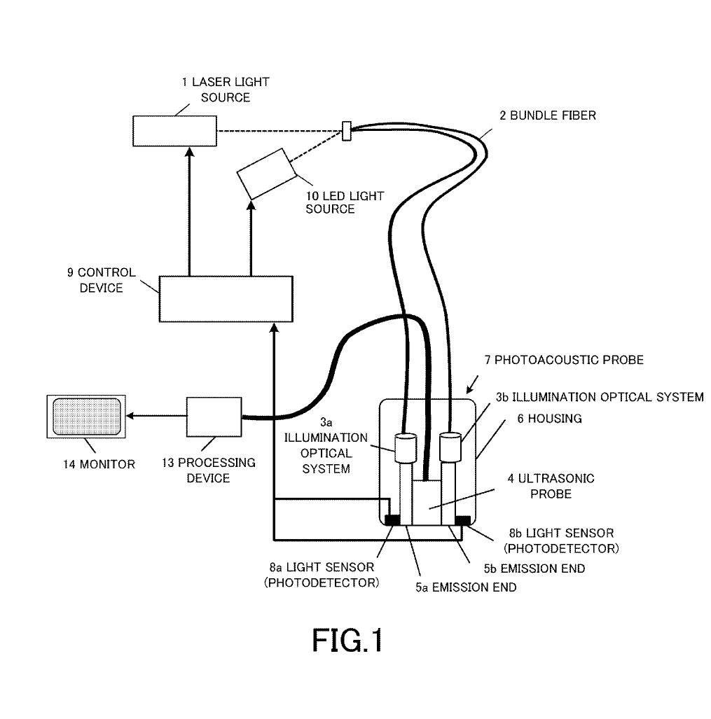

First, the configuration of a photoacoustic measurement apparatus according to a first embodiment is explained with reference to FIG. 1. The photoacoustic measurement apparatus according to the first embodiment of the present invention is a photoacoustic imaging apparatus that images information concerning a living organism, which is an object, for the purpose of a diagnosis of a malignant tumor, a vascular disease, and the like, follow-up of a chemical treatment, and the like. The information concerning the living organism is a generation source distribution of an acoustic wave caused by light irradiation and is an initial sound pressure distribution in the living organism or an optical energy absorption density distribution derived from the initial sound pressure distribution.

The photoacoustic measurement apparatus according to the first embodiment of the present invention is configured from a laser light source 1, a bundle fiber 2, a photoacoustic probe 7, a control device 9, an LED light source 10, a processing device 13, and a monitor 14. An overview is explained below concerning a method of measuring an object while respective units configuring the photoacoustic measurement apparatus according to the first embodiment are explained.

<<Laser Light Source 1>>

The laser light source 1 is a unit for generating a near infrared ray to be irradiated on a living organism, which is an object.

It is preferable to generate, from the laser light source 1, light having a specific wavelength absorbed by a specific component among components forming the living organism. Specifically, a pulse light source capable of generating pulse light in several nanosecond to several hundred nanosecond order is preferable. Although a laser is preferable as a light source, a light-emitting diode or the like can also be used instead of the laser. When the laser is used, various lasers such as a solid-state laser, a gas laser, a dye laser, and a semiconductor laser can be used.

The laser light source 1 is the first light source in the present invention. The laser light emitted by the laser light source 1 is the first irradiation light in the present invention.

Note that, although a single light source is used in this embodiment, a plurality of light sources may be used. When the plurality of light sources are used, in order to increase irradiation intensity of light irradiated on the living organism, a plurality of light sources that oscillate at the same wavelength may be used. In order to measure a difference due to a wavelength of an optical feature value distribution, a plurality of light sources having different oscillation wavelengths may be used. Note that, if a dye or OPO (Optical Parametric Oscillators) capable of converting an oscillating wavelength is used as the light source, it is also possible to measure the difference due to the wavelength of the optical feature value distribution.

A wavelength in use is preferably in a region of 700 nm to 1100 nm where absorption is little in a living organism. However, when an optical feature value distribution of a living organism tissue relatively close to the surface of the living organism is calculated, it is also possible to use a wavelength region wider than the wavelength region explained above, for example, a wavelength region of 400 nm to 1600 nm. It is preferable to select a specific wavelength among lights in the range according to a component to be measured.

Usually, an irradiation frequency of the laser light source is fixed. This is set as a setting value in order to continuously irradiate pulse light having desired intensity. Since the irradiation frequency affects the number of times of photoacoustic measurement that can be performed in a unit time, a higher irradiation frequency is more preferable. In this embodiment, the irradiation frequency of the laser light source is set to 10 Hz.

Light emitted by the laser light source 1 (hereinafter, laser light) is guided to the photoacoustic probe 7 by the bundle fiber 2 connected to the laser light source 1.

<<Bundle Fiber 2>>

The bundle fiber 2 is an aggregate of optical fibers for guiding the laser light generated by the light source to the photoacoustic probe 7. In this embodiment, a bundle fiber is used for transmission of the laser light. However, the transmission of the laser light may be performed by a combination of a light blocking tube and a reflecting mirror. Anything may be used as long as the laser light emitted by the light source can reach the photoacoustic probe. In this embodiment, since there are two systems as illumination optical systems, incident laser light is distributed to the two systems by the bundle fiber.

The configuration of the photoacoustic probe 7 is explained. The photoacoustic probe 7 is configured from a housing 6 and illumination optical systems 3a and 3b, an ultrasonic probe 4, emission ends 5a and 5b, and light sensors 8a and 8b housed in the housing 6.

Note that, in the explanation of the embodiment, an illumination optical system 3 is a collective term for the illumination optical systems 3a and 3b, an emission end 5 is a collective term for the emission ends 5a and 5b, and a light sensor 8 is a collective term for the light sensors 8a and 8b.

<<Illumination Optical System 3>>

The illumination optical systems 3a and 3b are units for performing beam formation of incident laser light. Specifically, the illumination optical systems 3a and 3b are optical members configured by lenses and diffusers such that a desired beam shape and a desired light intensity distribution can be obtained. In this embodiment, the illumination optical systems 3a and 3b are configured by expansion optical systems for expanding an illumination range and diffusers for preventing a sudden laser intensity distribution.

In this embodiment, the illumination optical systems are arranged on the inside of the photoacoustic probe 7. However, a configuration in which the illumination optical systems are arranged further on the light source side than the bundle fiber 2 may be adopted or a configuration in which the illumination optical systems are arranged in a plurality of places and a beam forming process is performed dividedly for the plurality of places may be adopted.

The formed laser light is emitted from the emission ends 5a and 5b, which are opening sections provided in the housing 6 (the emission ports in the present invention), and irradiated on an object.

When the irradiated laser light diffuses in the object and a part of the energy of the light propagated on the inside of the object is absorbed by a light absorber such as a blood vessel, an acoustic wave is generated from the light absorber by thermal expansion. That is, the light absorber absorbs the laser light, whereby the temperature of the light absorber rises. As a result, volume expansion occurs and an acoustic wave is generated. This phenomenon is generally referred to as photoacoustic effect. The acoustic wave is typically an ultrasound wave and includes acoustic waves called a sound wave, an ultrasound wave, a photoacoustic wave, a light-induced ultrasound wave, and the like.

<<Ultrasonic Probe 4>>

The ultrasonic probe 4 is a unit for detecting an acoustic wave generated or reflected on the inside of a living organism, which is an object, and converting the acoustic wave into an analog electric signal. The acoustic wave generated from the living organism is an ultrasound wave of 100 KHz to 100 MHz. Therefore, an ultrasonic detector that can receive the frequency band is used for the ultrasonic probe 4. Specifically, the ultrasonic detector is a transducer that makes use of a piezoelectric phenomenon, a transducer that makes use of resonance of light, a transducer that makes use of a change in a capacity, or the like. Any detector may be used as long as an acoustic wave signal can be detected.

The electric signal converted by the ultrasonic probe 4 is converted into image data by the processing device 13. It is possible to visualize object information by acquiring and analyzing the photoacoustic wave in this way. The object information to be visualized is a generation source distribution of the photoacoustic wave inside the object, an initial sound pressure distribution in the object, an optical energy absorption density distribution and a light absorption coefficient distribution derived from the initial sound pressure distribution, and a concentration distribution of a substance forming a tissue. The concentration distribution of the substance is, for example, an oxygen saturation distribution or an oxidation-reduction hemoglobin concentration distribution. The generated image data is displayed on the monitor 14 and presented to a user.

<<LED Light Source 10>>

The photoacoustic measurement apparatus according to this embodiment further includes the LED light source 10 in addition to the components explained above. The LED light source 10 is a light source configured to emit light for evaluation (hereinafter, evaluation light) that simulates the laser light. In this embodiment, it is determined using the evaluation light to which degree the laser light leaks to the outside of the photoacoustic probe (hereinafter simply referred to as probe). The LED light source 10 is the second light source in the present invention. The evaluation light emitted by the LED light source 10 is the second irradiation light in the present invention.

Since the evaluation light is irradiated prior to the irradiation of the laser light, the LED light source 10 is desirably a light source that emits light of an energy level safe for the human body. The evaluation light source is not limited to the LED and other light emitting systems such as a solid-state laser, a liquid laser, and a gas laser may be used as long as the light emitting systems are permitted in terms of safety.

Like the laser light, the evaluation light is arranged to be guided by the bundle fiber 2. In this embodiment, the laser light and the evaluation light share the same bundle fiber. Therefore, both of incident angles of optical axes from the LED light source 10 and the laser light source 1 on the bundle fiber 2 are within a critical angle of the bundle fiber 2.

Note that, when the wavelengths of the laser light and the evaluation light are greatly different, an aberration or the like that occurs in the illumination optical system affects the wavelengths. Therefore, it is preferable to set the wavelength of the evaluation light emitted by the LED light source 10 to wavelength as close as possible to the wavelength of the laser light emitted by the laser light source 1.

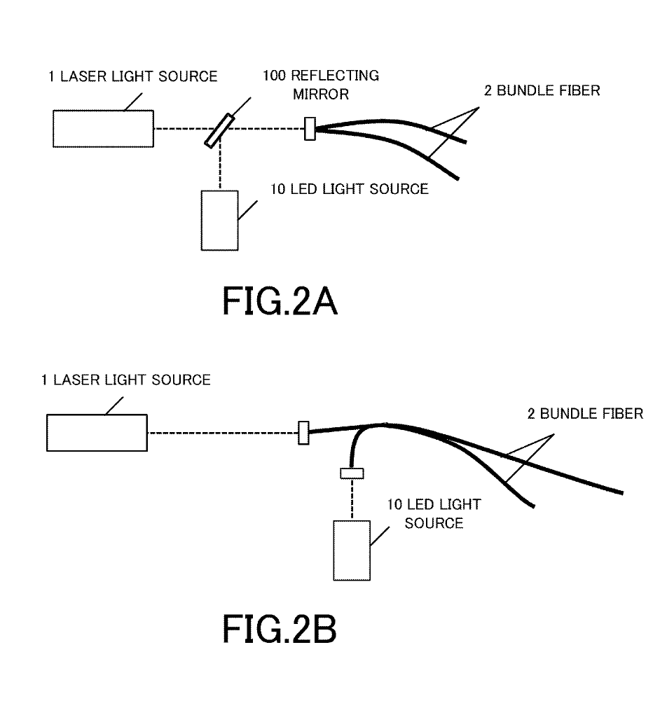

Concerning a method of merging the optical axes of the laser light and the evaluation light, as shown in FIG. 2A, a reflecting mirror may be used or, as shown in FIG. 2B, the bundle fiber 2 having a forked inlet may be used to merge the optical axes in the bundle fiber.

A polarized beam splitter or a cold mirror can be used for a reflecting mirror 100 used in FIG. 2A. When the polarize beam splitter is used, it is possible to merge the optical axes by applying polarization for transmitting the laser light through the splitter to the laser light and applying polarization for reflecting the evaluation light to the evaluation light. In this embodiment, since the laser light is an infrared ray, it is possible to merge the optical axes using a cold mirror or a hot mirror by setting the wavelength of the evaluation light in a visible ray region.

Like the laser light, the evaluation light is distributed to the two systems by the bundle fiber and made incident on the illumination optical systems 3a and 3b. Then, through beam formation same as the beam formation of the laser light, the evaluation light is emitted from the emission ends 5a and 5b and irradiated on the object.

In this embodiment, since the irradiation of the laser light is reproduced using the evaluation light, it is important that the evaluation light by the light source for evaluation causes a light amount distribution similar to a light amount distribution of the laser light near the surface of the object. Therefore, the laser light and the evaluation light share the same illumination optical system. That is, the illumination optical system 3 is the first illumination optical system and the second illumination optical system in the present invention.

In this embodiment, the LED light source 10 is arranged further on the laser light source 1 side, that is, on the main body side than the bundle fiber 2. However, the LED light source 10 may be arranged in the probe. In that case, the evaluation light is directly made incident on the illumination optical system 3 by the reflecting mirror not via the fiber. A system for arranging a branch point of the bundle fiber shown in FIG. 2B and the LED light source 10 in the probe and merging the optical axes from a halfway portion of the bundle fiber 2 may be adopted.

<<Light Sensor 8>>

The light sensors 8a and 8b are sensors respectively arranged near the emission ends 5a and 5b in order to detect the evaluation light. The positions and the wavelengths to be detected of the light sensors 8a and 8b are tuned and a plurality of the light sensors 8a and a plurality of the light sensors 8b are arranged near the emission ends 5a and 5b such that intensity signals of the evaluation light diffused in the object and transmitted through the object and the evaluation light reflected on the surface of the object can be acquired.

<<Control Device 9>>

The control device 9 is a unit for determining propriety of irradiation of the laser light on the basis of the intensity signal of the reflected light obtained by the light sensors 8a and 8b. Specific processing content is explained later.

<Irradiation Processing for the Laser Light>

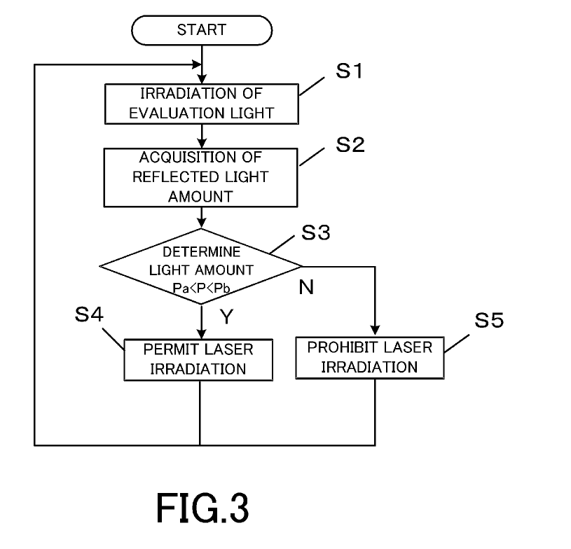

Laser light irradiation processing performed by the photoacoustic measurement apparatus according to this embodiment is explained below with reference to a flowchart shown in FIG. 3. Processing in steps S1 to S5 is processing for irradiating the evaluation light to thereby predict a leak of the laser light, which occurs between the probe and the object, and determining propriety of irradiation of the laser light. Since the laser light source 1 periodically emits light, the processing shown in FIG. 3 is also executed periodically. Specifically, the processing is repeatedly executed after the last light emission of the laser light ends until the next light emission is performed.

First, in step S1, the control device 9 controls the LED light source 10 and irradiates the evaluation light on the object.

In step S2, the control device 9 acquires a light intensity value of the reflected light from the intensity signal of the reflected light acquired by the light sensors 8a and 8b. The obtained light intensity value is a reflected light amount of the evaluation light reflected in an illumination region (a region where the evaluation light is irradiated on the object).

Subsequently, in step S3, the control device 9 determines propriety of laser light irradiation on the basis of the light intensity value. Note that, in this embodiment, since there are two systems as illumination optical systems, the determination of propriety of the laser light irradiation is performed for each of the two systems.

Concerning the determination processing performed in step S3, that is, the processing for determining propriety of the laser irradiation on the basis of the light intensity value of the reflected evaluation light, the idea of the processing and an example of implementation of the processing is explained.

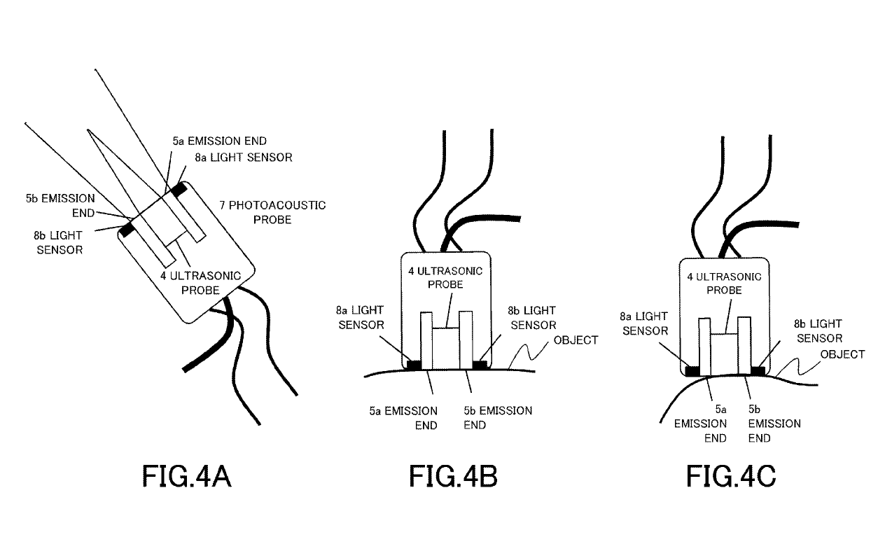

Light received by the light sensors 8a and 8b is the evaluation light emitted from the LED light source 10 and reflected in the illumination region. As contact patterns of the probe and the object, three patterns shown in FIG. 4 are illustrated.

FIG. 4A represents a pattern in which the probe is completely separated from the object. In this case, the irradiated evaluation light does not return by being reflected or only light reflected in the distance and diffused returns. That is, the intensity of light detected by the light sensors 8a and 8b is an extremely small value.

FIG. 4B represents a pattern in which the probe and the object sufficiently adhere to each other. In this case, the irradiated evaluation light is diffused and reflected on the inside of the object. Therefore, the intensity of light detected by the light sensors 8a and 8b is a small value with respect to the irradiation intensity of the evaluation light. However, the value is a sufficiently large value compared with the intensity in the case of the pattern shown in FIG. 4A.

Lastly, FIG. 4C represents a pattern in which apart of the probe is lifted from the object. In this case, a part of light reflected in the object is detected. However, light reflected on the surface of the object is mainly detected. The intensity of the light reflected on the surface of the object is a sufficiently large value with respect to the reflected light in the object (the pattern shown in FIG. 4B). The same applies when the entire probe is slightly lifted from the object.

In order to determine propriety of irradiation of the laser light concerning each of the three patterns, two values described below are used as predetermined thresholds.

Threshold 1 (hereinafter, P1): A lower limit value that an intensity value of the evaluation light reflected on the inside the object can take

Threshold 2 (hereinafter, P2): A value based on an upper limit value of laser light intensity permitted in terms of safety

P1 is a value representing an intensity value of the evaluation light reflected on the inside of the object when the evaluation light is irradiated in a state in which the probe adheres to the object. The intensity of the light reflected in the object is different depending on a measurement segment and the age and the like of a test subject. Therefore, as P1, it is preferable to adopt a smallest value in a range of assumed intensity of the reflected light.

An example of a setting value of P2 is explained.

As an index of the intensity of light that can be irradiated on a living organism, there is a maximal permissible exposure (MPE) that can be irradiated on the surface of the living organism. A specific value of the MPE is specified in 60825-1 “Safety of laser products” of the International Electro-technical Commission (IEC). The value is also specified in JIS C 6802 “Safety Standard for Laser Products” of the Japan Industrial Standard (JIS) conforming to the IEC.

The MPE is a maximum value of radiation illuminance, which is a radiation amount per unit area, and designated for each segment of the human body. In particular, since retinas are susceptible to influence compared with the other tissues, a strict MPE value is set for the retinas. A probe of a manual scanning type can freely change an emitting direction of the laser light. Therefore, it is preferable to adopt, as the threshold P2, a most strict value among MPEs for the human body. In this embodiment, a setting value of P2 is set on the basis of the MPE for the retinas.

Note that the light sensors 8a and 8b receive the evaluation light rather than the laser light. Therefore, it is necessary to set P2 by converting the irradiation intensity of the evaluation light into irradiation intensity of the laser light used for measurement.

In this embodiment, the evaluation light and the laser light share the illumination optical system. Therefore, since light amount distributions near the object are similar, alight amount can be converted simply by multiplying the light amount with a coefficient.

When a ratio of irradiation energies of the evaluation light and the laser light is 1:N, an MPE that damages the retinas under irradiation conditions according to the embodiment is M [J/m²], and a light receiving area of the light sensors is S [m²], a value of P2 is calculated as indicated by Expression 1.

$$\begin{matrix}[ Math . 1 ] & \\ P 2 = M \cdot \frac{S}{N} [ J ] & ( Expression 1 )\end{matrix}$$

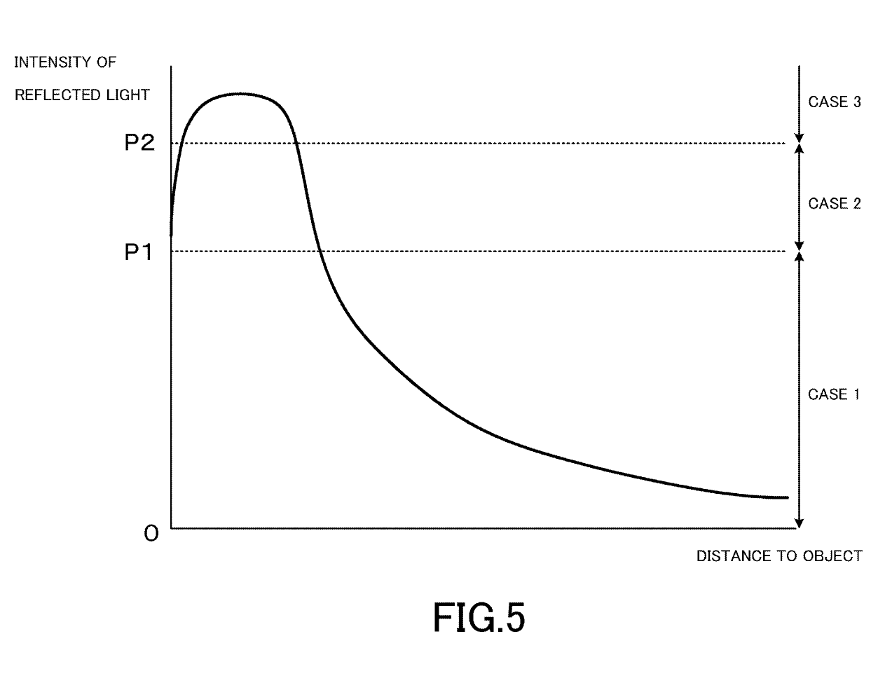

A graph of a relation between intensity P of the reflected light detected by the light sensors 8a and 8b and the distance between the probe and the object is FIG. 5. When the intensity of the detected reflection light is P, an irradiation propriety determination condition for the laser light is as described below.

P<=P1 (Case 1)

It is determined that the probe is completely lifted from the object. Control for prohibiting irradiation of the laser light is performed.

P1<P<P2 (Case 2)

It is determined that the probe completely adheres to the object or, although there is a gap, leak light is in a safe level. Control for permitting irradiation of the laser light is performed.

P>=P2 (Case 3)

It is determined that, although the probe and the object are close to each other, the probe is lifted from the object and an irradiation leak of a high level occurs. Control for prohibiting irradiation of the laser light is performed.

In this embodiment, since there are the two systems as the illumination optical systems and as the light sensors, the irradiation propriety determination for the laser light is performed for each of the systems. When it is determined that irradiation of the laser light is prohibited in one of the systems, the control device 9 controls the laser light source 1 to stop light emission.

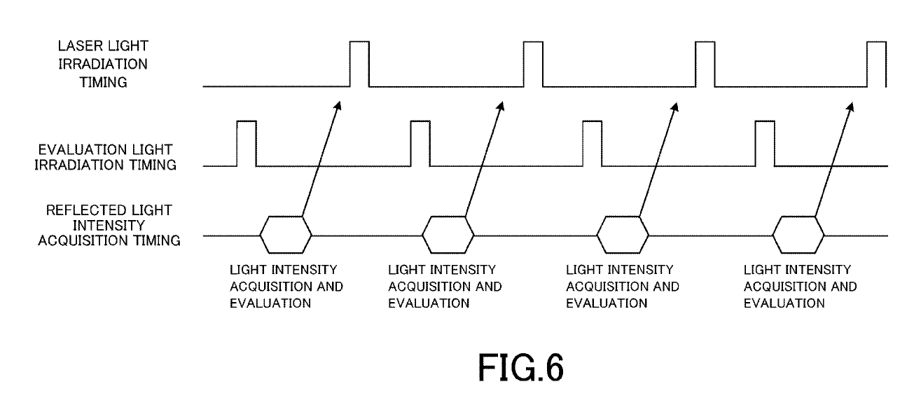

FIG. 6 is a diagram representing timing for emitting the laser light and the evaluation light when the control explained above is performed. Irradiation of the evaluation light is performed every time before irradiation of the laser light is performed. Only when the control device 9 determines that the laser light may be emitted, the irradiation of the laser light at the cycle is performed.

As explained above, in the photoacoustic measurement apparatus according to this embodiment, since the laser light for measurement and the evaluation light for detecting leak light pass through the same illumination optical system, it is possible to irradiate the evaluation light that faithfully simulates the laser light for measurement. Consequently, it is possible to faithfully reproduce a reflection state of the laser light in the illumination region and accurately estimate a leak of the laser light. Therefore, it is possible to dramatically improve safety of the apparatus. Since the evaluation light is light of a safe energy level, there is an advantage that it is unnecessary to pay attention to handling.

In a photoacoustic measuring apparatus of a manual scanning type, measurement efficiency is pursued. Therefore, the size of an ultrasonic probe and the aperture size of an illumination optical system increase and a probe tends to be increased in size. On the other hand, there is also a segment such as the armpit of the human body where it is difficult to place the probe to completely adhere to the body depending on a measurement segment. If irradiation propriety is determined using a contact sensor or the like, such a case cannot be dealt with. However, the photoacoustic measurement apparatus according to this embodiment can carry out measurement if a leak of the laser light to the outside of the probe is sufficiently small.

In this way, the photoacoustic measurement apparatus according to this embodiment performs irradiation control of the laser light according to whether leak light is in a safe level rather than whether the probe adheres to the object. Therefore, it is possible to attain both of safety and improvement of measurement efficiency.

Note that the object explained in the first embodiment is a living organism such as a breast. However, the present invention can also be applied to an object information acquiring apparatus that measures various objects other than the living organism. In the first embodiment, the two systems of the illumination optical systems and the light sensors are used. However, an arbitrary number of the illumination optical systems and the light sensors can be used.

In the first embodiment, the laser light and the evaluation light are irradiated on the object through the same illumination optical system. However, only a part of the illumination optical system may be shared. For example, a diffuser for merging the routes of the laser light and the evaluation light and equalizing an intensity distribution of the laser may be shared. The laser light and the evaluation light may be emitted from different emission ends after light beams are formed by the same illumination optical system. The illumination optical system may be shared in any way as long as an irradiation range and a light amount distribution of the laser light can be simulatively reproduced by the evaluation light.

### Second Embodiment

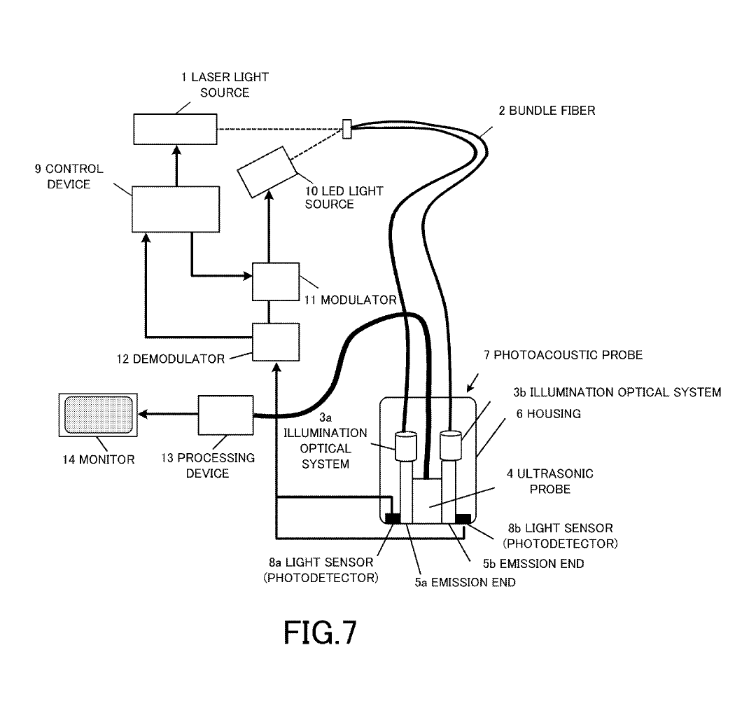

In the first embodiment, the reflection of the evaluation light is detected by the light sensor. However, since ambient environmental light is made incident on the light sensor together with the reflected light, there is a problem in that an accurate reflected light amount cannot be obtained depending on the wavelength of the evaluation light. A second embodiment is an embodiment for applying modulation and demodulation to the evaluation light to deal with the problem. A configuration diagram of a photoacoustic measurement apparatus according to the second embodiment is shown in FIG. 7. Units same as those in the first embodiment are denoted by the same reference numerals and signs and explanation of the unit is omitted.

The photoacoustic measurement apparatus according to the second embodiment is different from the first embodiment in that the photoacoustic measurement apparatus further includes a modulator 11 and a demodulator 12.

The modulator 11 is a unit for applying modulation to evaluation light emitted by an LED light source 10. As a pattern of the modulation, an arbitrary pattern can be used. The demodulator 12 is a synchronous demodulator and is a unit for demodulating an input signal according to a modulation pattern same as the modulation pattern of the modulator 11. That is, the LED light source 10 and the modulator 11 configure the second light source, and the light sensor 8 and demodulator 12 configure a light sensor unit according to the present invention.

In the second embodiment, when the control device 9 performs determination of a light intensity value in step S3, the control device 9 performs the determination targeting a signal demodulated by the demodulator 12. That is, a signal of light not modulated by the modulator 11 is not a target of the determination because a modulation pattern of the light does not match. Therefore, even when the ambient environment light is made incident on the light sensor 8, it is possible to extract only a signal corresponding to the evaluation light irradiated from the LED light source 10 and perform the determination.

In the second embodiment, the modulation is applied to the evaluation light and the determination is performed using only an intensity signal of light demodulated according to the same pattern in this way. Consequently, it is possible to set a wavelength of a light source for evaluation to a wavelength in the same region as the laser light for measurement and the environment light. Therefore, it is possible to obtain an advantage that a degree of freedom of selection increases.

Note that the explanation of the embodiments is illustration in explaining the present invention. The present invention can be carried out by changing or combining the embodiments as appropriate without departing from the spirit of the invention. The present invention can be carried out as a control method for the object information acquiring apparatus including at least apart of the processing explained above or can be carried out as a program for causing the object information acquiring apparatus to execute these methods. The processing and the units explained above can be freely combined and carried out as long as a technical inconsistency does not occur.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-208123, filed on Sep. 21, 2012, which is hereby incorporated by reference herein in its entirety.

### REFERENCE SIGNS LIST

1 Laser light source, 10 LED light source, 3 Illumination optical system, 8 Light sensor, 9 Control device

1. An object information acquiring apparatus that irradiates light on an object, receives an acoustic wave generated in the object, and acquires information concerning an inside of the object on the basis of the acoustic wave, the object information acquiring apparatus comprising:
a first light source configured to generate first irradiation light to be irradiated on the object, this light being emitted to generate an acoustic wave from the object;
a second light source configured to generate second irradiation light to be irradiated on the object;
a first illumination optical system connected to said first light source and configured to guide the first irradiation light to the object;
a second illumination optical system connected to said second light source and configured to guide the second irradiation light to the object;
a light sensor unit including a light sensor and configured to acquire an intensity signal of reflected light on the object resulting from the reflection of the second irradiation light irradiated on the object; and
a control device configured to determine irradiation of the first irradiation light on the basis of the intensity signal of the reflected light acquired by said light sensor unit,
wherein said second illumination optical system shares at least a part of an optical member with said first illumination optical system.

2. The object information acquiring apparatus according to claim 1, wherein, when the intensity signal of the reflected light acquired by said light sensor unit indicates a value in a predetermined range, said control device enables irradiation of the first irradiation light.

3. The object information acquiring apparatus according to claim 2, wherein an upper limit value of the predetermined range is a value indicating that reflected light resulting from the reflection of the first irradiation light on a surface of the object has an intensity that cannot be permitted in terms of safety.

4. The object information acquiring apparatus according to claim 1, wherein an emission port of the first irradiation light and an emission port of the second irradiation light are the same.

5. The object information acquiring apparatus according to claim 1, wherein
said second light source further includes a modulation unit configured to modulate the second irradiation light according to a predetermined modulation pattern, and
said light sensor unit acquires a signal matching the modulation pattern from an intensity signal of light detected by said light sensor thereby acquiring the intensity signal of the second irradiation light that has been reflected by the object.

6.-10. (canceled)

11. The object information acquiring apparatus according to claim 1, wherein a lower limit value of the predetermined range is a value that can be taken by the evaluation light reflected on the inside of the object.

12. The object information acquiring apparatus according to claim 1, wherein said second illumination optical system shares at least a part of an optical member with said first illumination optical system so that the second irradiation light causes a light amount distribution similar to a light amount distribution of the first irradiation light near the surface of the object.

13. The object information acquiring apparatus according to claim 12, wherein,
when the intensity signal of the reflected light acquired by said light sensor unit indicates a predetermined range, said control device enables irradiation of the first irradiation light, and
when a ratio of irradiation energies of the second irradiation light and the first irradiation light is 1:N, a maximal permissible exposure that damages is M [J/m²], and a light receiving area of the light sensor is S [m²], an upper limit value of the predetermined range is indicated by Expression 1:
$$P 2 = M \cdot \frac{S}{N} [ J ] \qquad ( Expression 1 )$$

14. The object information acquiring apparatus according to claim 13, wherein the maximal permissible exposure is specified in 60825-1 “Safety of laser products” of the International Electro-technical Commission.

15. The object information acquiring apparatus according to claim 1, wherein, when the intensity signal of the reflected light acquired by said light sensor unit does not indicate a value in a predetermined range, said control device prohibits irradiation of the first irradiation light.

16. The object information acquiring apparatus according to claim 1, wherein said light sensor unit comprises a plurality of light sensors, and
wherein said control device determines irradiation of the first irradiation light based on the intensity signal of the reflected light acquired by said plurality of light sensors respectively.

17. The object information acquiring apparatus according to claim 1, wherein said light sensor and at least a part of said first illumination optical system are arranged in a probe, and
wherein said first illumination optical system has an emission port, and said light sensor is arranged on outward from said emission port of said first illumination optical system with respect to the center of said probe.

18. A control method for an object information acquiring apparatus that irradiates light on an object, receives an acoustic wave generated in the object, and acquires information concerning an inside of the object on the basis of the acoustic wave, the control method comprising:
a step of generating first irradiation light to be irradiated on the object, with this light being emitted to generate an acoustic wave from the object, and of irradiating the first irradiation light on the object through an illumination optical system;
a step of generating second irradiation light to be irradiated on the object and irradiating the second irradiation light on the object through at least a part of the illumination optical system through which the first irradiation light is irradiated; and
a step of acquiring, by a light sensor, an intensity signal of reflected light on the object resulting from the reflection of the second irradiation light irradiated on the object, and of determining irradiation of the first irradiation light on the basis of the acquired intensity signal.

19. The control method for an object information acquiring apparatus according to claim 18, wherein, in said step of performing irradiation control of the first irradiation light, when the intensity signal of the reflected light indicates a value in a predetermined range, irradiation of the first irradiation light is enabled.

20. The control method for an object information acquiring apparatus according to claim 19, wherein an upper limit value of the predetermined range is a value indicating that reflected light on a surface of the object resulting from the reflection of the first irradiation light has an intensity that cannot be permitted in terms of safety.

21. The control method for an object information acquiring apparatus according to claim 18, wherein, in said step of irradiating the second irradiation light, the second irradiation light is irradiated on the object from an emission port from which the first irradiation light also is irradiated.

22. The control method for an object information acquiring apparatus according to claim 18, wherein
in said step of irradiating the second irradiation light, the second irradiation light is modulated according to a predetermined modulation pattern, and
in said step of performing irradiation control of the first irradiation light, a signal matching the modulation pattern is extracted from an intensity signal of light detected by the light sensor to acquire the intensity signal of the reflected light on the object resulting from the reflection of the second irradiation light.

23. The control method for an object information acquiring apparatus according to claim 18, wherein a lower limit value that can be taken by of the predetermined range is a value of the evaluation light reflected on the inside of the object.

24. The control method for an object information acquiring apparatus according to claim 18, wherein, in said step of irradiating the second irradiation light, the second irradiation light is irradiated on the object through at least a part of the illumination optical system through which the first irradiation light is irradiated so that the second irradiation light causes a light amount distribution similar to a light amount distribution of the first irradiation light near the surface of the object.

25. The object information acquiring apparatus according to claim 24, wherein,
wherein, in said step of performing irradiation control of the first irradiation light, when the intensity signal of the reflected light indicates a value in a predetermined range, irradiation of the first irradiation light is enabled, and
when a ratio of irradiation energies of the second irradiation light and the first irradiation light is 1:N, a maximal permissible exposure that damages is M [J/m²], and a light receiving area of the light sensor is S [m²], an upper limit value of the predetermined range is indicated by Expression 1:
$$P 2 = M \cdot \frac{S}{N} [ J ] \qquad ( Expression 1 )$$

26. The object information acquiring apparatus according to claim 25, wherein the maximal permissible exposure is specified in 60825-1 “Safety of laser products” of the International Electro-technical Commission.

27. The object information acquiring apparatus according to claim 18, wherein, in said step of performing irradiation control of the first irradiation light, when the intensity signal of the reflected light does not indicate a value in a predetermined range, irradiation of the first irradiation light is prohibited.

28. An object information acquiring apparatus that irradiates light on an object, receives an acoustic wave generated in the object, and acquires information concerning an inside of the object on the basis of the acoustic wave, the object information acquiring apparatus comprising:
a first light source configured to generate first irradiation light to be irradiated on the object, this light being emitted to generate an acoustic wave from the object;
a second light source configured to generate second irradiation light to be irradiated on the object;
a first illumination optical system connected to said first light source and configured to guide the first irradiation light to the object;
a second illumination optical system connected to said second light source and configured to guide the second irradiation light to the object;
a light sensor unit including a light sensor and configured to acquire an intensity signal of reflected light on the object resulting from the reflection of the second irradiation light irradiated on the object; and
a control device configured to control irradiation of the first irradiation light on the basis of the intensity signal of the reflected light acquired by said light sensor unit.
